(19) United States
(12) Patent Application Publication
(10) Pub. No.: US 2022/0291248 A1
(43) Pub. Date: Sep. 15, 2022

(54) OPTIMIZING LAB SPECIMEN FROM COLLECTION TO UTILIZATION

(71) Applicant: Optum, Inc., Minnetonka, MN (US)

(72) Inventors: Jon Kevin Muse, Thompsons Station, TN (US); Gregory J. Boss, Saginaw, MI (US); Komal Khatri, Cedar Park, TX (US); Lisa Jo L Abbo, Chicago, IL (US)

(21) Appl. No.: 17/196,484

(22) Filed: Mar. 9, 2021

(51) Int. Cl. G01N 35/00; B01L 3/00

(52) U.S. Cl. CPC G01N 35/00732; G01N 35/0092; B01L 3/508; G01N 2035/00346; G01N 2035/0094; B01L 2300/024; B01L 2300/025; B01L 2200/14; G08B 5/36

(57) ABSTRACT

A system includes sample containers and a computing system. The sample containers include one or more data storage media configured to store sample information relating to the respective sample contained in each sample container. The sample information include data indicating a sample type, an intended use of the sample contained within the respective sample container, and a sample collection time. The computing system is configured to receive the sample information from the sample containers and determine, based on the sample information, an action for increasing a likelihood that one or more applicable samples of the plurality of samples will be viable for the intended uses of the applicable samples. The computing system is further configured to perform the action.

### BACKGROUND

A sample (e.g., fluid, a tissue, an organ, etc.) intended to be used for a particular purpose may be collected from a subject (e.g., a person, an animal, a plant, etc.) and placed in a sample container. Following collection of the sample, the sample may deteriorate (e.g., due to the passage of time, environmental conditions, etc.), potentially reducing the period of viability during which the sample may be used for its intended use. If the period of viability elapses, a replacement for the sample may need to be obtained. However, obtaining another sample may be difficult (e.g., due to the scarcity of the sample), untimely (e.g., due to the urgency of the use of the sample), and/or the like.

In some instances, multiple samples may need to be transported elsewhere (e.g., a laboratory). The samples may have varying periods of viability, such that some samples may expire before others. Accordingly, unless the viability of each sample is being monitored, a delivery of the samples may be suitable for some samples but not other samples. That is, the delivery may result in the period of viability elapsing for some of the samples. This result may be costly and even dangerous (e.g., to a patient from whom the sample was collected).

### SUMMARY

In general, this disclosure is directed to techniques for monitoring and increasing the viability of samples. For example, each sample container of a plurality of sample containers may include data storage media configured to store sample information. Based on the sample information, a computing system may determine an action for increasing a likelihood that samples that are important (e.g., based on the scarcity of the sample, the urgency of the use of the sample, etc.) will be viable for their intended use. In this way, the techniques described here may enable a computing system to efficiently collect sample information and effectively act thereupon to ensure the viability of samples, particularly important ones. Thus, the techniques may better ensure the viability of samples compared to other approaches.

In some examples, a system includes: a plurality of sample containers; a storage container defining a cavity for storing the plurality of sample containers, wherein: each respective sample container of the plurality of sample containers defines a cavity to contain a respective sample of a plurality of samples, and each respective sample container of the plurality of sample containers includes one or more data storage media configured to store sample information relating to the respective sample contained in each sample container, the sample information including data indicating: a sample type; an intended use of the sample contained within the respective sample container; and a sample collection time; and a computing system configured to: receive the sample information from the sample containers; and determine, based on the sample information, an action for increasing a likelihood that one or more applicable samples of the plurality of samples will be viable for the intended uses of the applicable samples; and perform the action.

In some examples, a method includes: receiving, by a computing system, sample information from each respective sample container of a plurality of sample containers, wherein: each respective sample container defines a cavity to contain a respective sample of a plurality of samples, and each respective sample container of the plurality of sample containers comprises one or more data storage media configured to store the sample information relating to the respective sample contained in each sample container, the sample information comprising data indicating: a sample type; an intended use of the sample contained within the respective sample container; and a sample collection time; determining, by the computing system and based on the sample information, an action for increasing a likelihood that one or more applicable samples of the plurality of samples will be viable for the intended uses of the applicable samples; and performing, by the computing system, the action.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system for monitoring and increasing viability of samples in accordance with techniques of this disclosure.

FIG. 2 is a conceptual diagram illustrating an example storage container for storing a plurality of sample containers in accordance with techniques of this disclosure.

FIG. 3 is a block diagram illustrating an example system for monitoring and increasing viability of samples in accordance with techniques of this disclosure.

FIG. 4 is a flowchart illustrating an exemplary operation of an example system in accordance with techniques of this disclosure.

### DETAILED DESCRIPTION

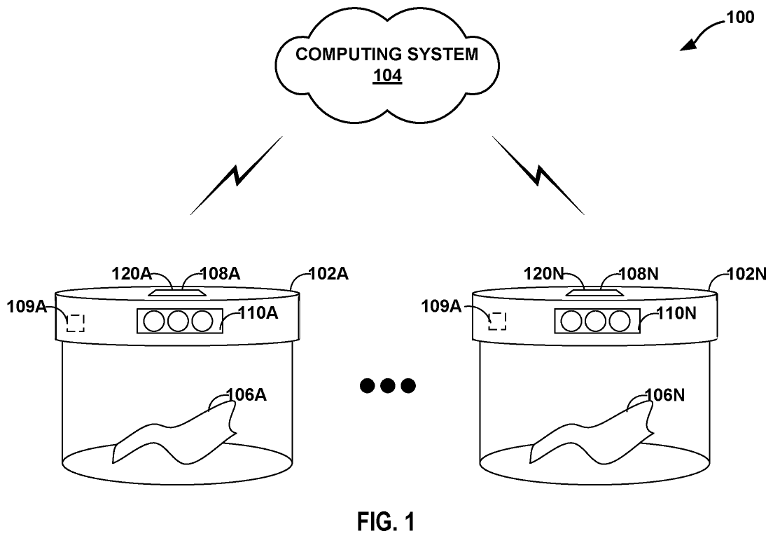

FIG. 1 is a conceptual diagram illustrating an example system 100 for monitoring and increasing viability of samples in accordance with techniques of this disclosure. As shown in the example of FIG. 1, system 100 includes a plurality of sample containers 102A-102N (collectively, “sample containers 102”) and a computing system 104. Sample containers 102 may be similar to one another. Thus, the description of one sample container (e.g., sample container 102A) may apply equally to the others (e.g., sample container 102B, sample container 102C, etc.).

Each of sample containers 102 may define a cavity to contain a respective sample (e.g., fluid, a tissue, an organ, etc.) of a plurality of samples 106A-106N (collectively, “samples 106”). Sample containers 102 may be bottles, vials, pots, tubes, or other types of containers made of plastic, glass, or other materials suitable for containing samples 106. Sample containers 102 may be leak-proof and sterile and may include identifiers (e.g., identification stickers). Sample containers 102 may be dimensioned to fit within receptacles (e.g., receptacles of a tray) of another container (e.g., a storage container).

Computing system 104 may be any suitable remote computing system, such as one or more desktop computers, laptop computers, mainframes, servers, cloud computing systems, virtual machines, and/or the like capable of sending and receiving information via a network. In some examples, computing system 104 may represent or include a cloud computing system that provides one or more services via a network. In some examples, computing system 104 may be a distributed computing system.

Samples 106 may be placed in sample containers 102 for an intended use. For example, samples 106 may need to be transported from one geographic location (e.g., a clinic) to another geographic location (e.g., a laboratory) to perform testing on samples 106. Following collection of samples 106, samples 106 may deteriorate (e.g., due to the passage of time, environmental conditions, etc.), potentially reducing the period of viability during which samples 106 may be used for their intended use. If the period of viability for a sample elapses, a replacement for the sample may need to be obtained. However, obtaining replacement samples may be difficult, untimely, and/or the like.

In accordance with techniques of this disclosure, system 100 may be configured to monitor each of samples 106 and perform one or more actions to increase a likelihood of applicable samples being viable for their intended uses. Each of sample containers 102 may include one or more data storage media 108A-108N (collectively, “data storage media 108”). For example, data storage media 108 may be embedded into, integrated into, appended to, attached to, extend from, or otherwise connected to one or more components (e.g., a base, a wall, a lid, etc.) of sample containers 102. In any case, data storage media 108 may be configured to store sample information. For each of sample containers 102, the sample information stored by the data storage media of a sample container may relate to the sample contained in the sample container.

The sample information may include information such as the type of the sample (e.g., the name of the sample, the properties of the sample, etc.), the intended use of the sample (e.g., the test to be performed on the sample), temperature conditions (e.g., data about the temperature at which the sample is to be stored, data about the temperature at which the sample has been stored, etc.), centrifuge detection (e.g., data about whether a centrifuge has been applied to the sample), a duration of viability (of the sample), light sensitivity (of the sample), sample container properties (e.g., whether the container is transparent, clear, dark, opaque, etc.), multi-variant date and time (e.g., origination of the sample (or, in other words, the time the sample was collected), the expiration date of the container, entries documenting when the sample container has been moved from one geographic location (e.g., a first laboratory) to another (e.g., a second laboratory), etc.), a sample importance score (discussed in greater detail below), electronic health record (EHR) data (e.g., name of the patient from whom the sample was collected, the date of birth of that patient, the name of the clinic that patient visits, the name of that patient's primary doctor, etc.), expected end-to-end process time (e.g., beginning with collection of the sample and ending with utilization of the sample), and/or the like.

As described above, the sample information may also include a sample importance score. The sample important scores may be used to determine which samples of samples 106 are applicable samples. The sample importance score may be based on various factors, each of which may be stored for each of samples 106 on the corresponding one or more of data storage media 108. For example, the factors may include the medical importance of the results from using the sample, the urgency of the results from using the sample, the collection importance of the sample (e.g., based on the difficulty of obtaining the sample, the availability of the sample, etc.), the handling importance (e.g., based on the sensitivity and/or fragility of the sample), and/or the like. The sample importance score may be calculated using an algorithm, a mathematical model, and/or other techniques.

Sample containers 102 may be configured to transmit the sample information stored on data storage media 108 to computing system 104. For example, each of sample containers 102 may include corresponding communication components 120A-120N (collectively, “communication components 120”). As shown in FIG. 1, communication components 120 may be included in the same circuitry (e.g., a printed circuit board (PCB) as data storage media 108. Alternatively, communication components 120 may be separate from data storage media 108.

Communication components 120 may be configured to receive and transmit various types of information, such as the sample information stored on data storage media 108, over a network. The network may include a wide-area network such as the Internet, a local-area network (LAN), a personal area network (PAN) (e.g., Bluetooth®), an enterprise network, a wireless network, a cellular network, a telephony network, a Metropolitan area network (e.g., WIFI, WAN, WiMAX, etc.), one or more other types of networks, or a combination of two or more different types of networks (e.g., a combination of a cellular network and the Internet).

Communication components 120 may include wireless communication devices capable of transmitting and/or receiving communication signals via the network, such as a cellular radio, a 3G radio, a 4G radio, a 5G radio, a Bluetooth® radio (or any other PAN radio), an NFC radio, or a WiFi radio (or any other WLAN radio). Additionally or alternatively, communication components 120 may include wired communication devices capable of transmitting and/or receiving communication signals via a direct link over a wired communication medium (e.g., a universal-serial-bus (“USB”) cable).

In some examples, sample containers 102 may include corresponding processing circuitry 109A-109N (collectively, “processing circuitry 109”). Processing circuitry 109 may be embedded into, integrated into, appended to, attached to, extend from, or otherwise connected to one or more components (e.g., a base, a wall, a lid, etc.) of sample containers 102. Processing circuitry 109 may be configured to implement functionality and/or execute instructions associated with sample containers 102 and/or computing system 104. In some examples, computing system 104 may include processing circuitry 109 of sample containers 102.

Computing system 104 may determine the sample importance of samples 106 based on the sample information received by computing system 104 from sample containers 102 (via communication components 120 of sample containers 102). For example, computing system 104 may determine the sample importance of samples 106 based on the medical importance of the results from using samples 106, the collection importance of samples 106, the handling importance of samples 106, etc.

Computing system 104 may classify samples that have relatively high sample importance scores as applicable samples. For example, computing system 104 may determine that sample 106A has a relatively high sample importance score because the medical importance of the results from using sample 106A is high, the urgency of the results from using sample 106A is high, the collection importance of sample 106A is high because obtaining sample 106A is difficult, and the handling importance of sample 106A is high because of the sensitivity of (the duration of viability of) sample 106A to the physical conditions of the environment in which sample 106A is stored. Accordingly, computing system 104 may classify sample 106A as an applicable sample.

Computing system 104 may not classify samples that do not have relatively high sample importance scores (or, in other words, samples that have relatively low sample importance scores) as applicable samples, computing system 104 may determine that sample 106B has a relatively low sample importance score because the medical importance of the results from using sample 106B is low, the urgency of the results from using sample 106B is low, the collection importance of sample 106B is low because obtaining sample 106B is easy, the handling importance of sample 106B is low because of the sensitivity of (the duration of viability of) sample 106B to the physical conditions of the environment in which sample 106B is stored. Accordingly, computing system 104 may not classify sample 106B as an applicable sample. By determining which samples of samples 106 are applicable samples in this way, computing system 104 may determine that the applicable samples have higher importance than other samples of samples 106 and that the viability of the applicable samples should be ensured, even at the expense of the viability of other samples.

Computing system 104 may be configured to increase a likelihood that one or more applicable samples of samples 106 will be viable for the intended uses of the applicable samples. That is, responsive to receiving the sample information from sample containers 102, computing system 104 may determine, based on the sample information, an action for increasing a likelihood that one or more applicable samples of samples 106 will be viable for the intended uses of the applicable samples and perform that action.

An example action that computing system 104 may perform to increase a likelihood that applicable samples will be viable for their intended use is controlling one or more physical conditions of an environment in which sample containers 102, and thus samples 106, are stored. For example, one or more physical conditions of an environment (e.g., a storage container configured to store and/or transport sample containers 102) in which sample containers 102, and thus samples 106, are stored may be conducive to the viability of one set of samples 106 without being conducive to the viability of another set of samples 106. Indeed, in some instances, the physical conditions of the environment may increase the viability of one set of samples 106 (e.g., samples 106A-106C) but decrease the viability of another set of samples 106 (samples 106D-106F). The physical conditions may include, but are not limited to, a temperature of the environment and a light level of the environment. Depending on the types of samples 106, such physical conditions may have a significant effect on the duration of viability, as illustrated by the following table:

Duration of Viability as a Function of Temperature

| Test Name | 21-25° C. | | 4-8° C. | | <−20° C. | |
|---|---|---|---|---|---|---|
| Adrenal Mass Panel, 24-hour, Urine | No | | 14 | days | 90 | days |
| Albumin, 24 Hour, Urine | 7 | days | 7 | days | 7 | days |
| Aldosterone, 24 Hour, Urine | 14 | days | 28 | days | 28 | days |
| Aldosterone with Sodium, 24 Hour, | 7 | days | 14 | days | 14 | days |
| Urine | | | | | | |
| Alpha-1-Microglobulin, 24 Hour, | 7 | days | 7 | days | 7 | days |
| Urine | | | | | | |
| Arsenic Speciation, 24 Hour, Urine | 72 | hours | 28 | days | 28 | days |

| | | | | |
|---|---|---|---|---|
| Arylsulfatase A, 24 Hour, Urine | No | 14 | days | No |

As shown in the above table, a duration of viability of samples 106 may vary depending on the temperature at which samples 106 are stored. For example, some samples (e.g., Arsenic Speciation, 24 Hour, Urine) have the longest duration of viability when stored in a 21-25° C. range (hereinafter referred to as “ambient temperatures”). Other samples (e.g., Arylsulfatase A, 24 Hour, Urine) have the longest duration of viability when stored in a 4-8° C. range (hereinafter referred to as “refrigerated temperatures”). Still other samples (e.g., Adrenal Mass Panel, 24-hour, Urine) have the longest duration of viability when stored in a range less than −20° C. (hereinafter referred to as “frozen temperatures”). Although not described herein, it should be understood that similar effects on the duration of viability of samples 106 may be seen with respect to other physical conditions of the environment in which samples 106 are stored.

As the ambient, refrigerated, and frozen temperature ranges are not overlapping, the temperature range of an environment in which sample containers 102 are stored may be conducive to the viability of one set of samples 106 without being conducive to the viability of another set of samples 106. For example, storing Adrenal Mass Panel, 24-hour, Urine in a frozen temperature range may allow for a duration of viability of Adrenal Mass Panel, 24-hour, Urine of 90 days. However, storing Arylsulfatase A, 24 Hour, Urine in a frozen temperature range may damage Arylsulfatase A, 24 Hour, Urine, potentially resulting in basically no duration of viability (e.g., 0 days).

Thus, it may be impossible to control the physical conditions of the environment in which samples 106 are stored to ensure the viability of all of samples 106. At the same time, not all of samples 106 may be of equal importance. Thus, it may be desirable to ensure the viability of the applicable samples (e.g., relatively important samples) even if that means allowing the viability of the remaining samples (e.g., relatively unimportant samples) to expire.

In the example above, computing system 104 may determine the temperature range of the environment in which samples 106 are stored that will increase the likelihood that the applicable samples will be viable for their intended uses and control the temperature range accordingly. For example, if the applicable samples include samples 106A-106C, and if computing system 104 determines (e.g., based on sample types, test names, etc.) that the temperature range that maximizes the viability of samples 106A-106C is the frozen temperature range, then computing system 104 may control the temperature range of the environment so the temperature range is the frozen temperature range. In some examples, computing system 104 may perform this action even if the frozen temperature range is not conducive to (e.g., damages) other samples (e.g., samples 106D-106F), potentially shortening the duration of viability of those other samples.

In a related example, if the applicable samples include samples 106D-106F, and if computing system 104 determines that the light level that maximizes the viability of samples 106D-106F is no light, then computing system 104 may control the light level of the environment so the light level is no light. In some examples, computing system 104 may perform this action even if no light is not conducive to the other samples (e.g., samples 106A-106C), shortening the duration of viability of those other samples. Although the discussion herein of physical conditions primarily relates to temperature and light level, it should be understood that controlling other physical conditions (e.g., humidity, air pressure, etc.) of the environment in a similar manner are contemplated by this disclosure.

As the supply chain for samples 106 may involve multiple stages, multiple parties, multiple locations, and/or the like, it may be difficult for one or more personnel to monitor and track samples 106 from collection to utilization. In turn, the personnel may fail to use samples 106 in a timely manner, potentially resulting in the duration of viability of one or more of samples 106 elapsing.

To increase the likelihood of the applicable samples being viable for their intended use, computing system 104 may determine and perform one or more actions in addition to or alternative to controlling the physical conditions of the environment in which samples 106 are stored. For example, computing system 104 may perform the action of communicating information to personnel involved in the supply chain for samples 106, potentially enabling the personnel to respond based upon the communicated information. In this way, the action of communicating information performed by computing system 104 may increase the likelihood that the applicable samples will be viable for their intended use.

In some examples, computing system 104 communicates information by causing sample containers 102 to output one or more indications. For example, each of sample containers 102 may include one or more indicators 110A-110N (collectively, indicators 110). Indicators 110 may be embedded into, integrated into, appended to, attached to, extend from, or otherwise connected to one or more components (e.g., a base, a wall, a lid, etc.) of sample containers 102.

In some examples, computing system 104 determines, based on the sample information received from sample containers 102, the duration of viability of samples 106. Computing system 104 may then cause indicators 110 to output an indication regarding viability of samples 106 contained in sample containers 102 for the intended use of samples 106. In some examples, indicators 110 may be light sources configured to display a plurality of indications. Each of the indications may correspond to a different duration of viability of samples 106 contained in sample containers 102.

For example, indicators 110 may display a first indication, a second indication, and a third indication. The first indication may correspond to a (remaining) duration of viability that is 90-100% of the maximum duration (or, in other words, the total expected duration) of viability of samples 106. The second indication may correspond to a duration of viability that is 10-90% of the maximum duration of viability of samples 106. The second indication may correspond to a duration of viability that is 0-10% of the maximum duration of viability of samples 106. It should be understood that the various indications may correspond to durations of viability other than the ones described herein.

The various indications (e.g., first indication, second indication, third indication, etc.) may be visually distinguishable such that a person who looks at indicators 110 may visually determine the (remaining) duration of viability of samples 106. For example, the various indications may be visually distinguishable based on color. For instance, the first indication may be a green light; the second indication may be a yellow light; the third indication may be a red light. In another example, the various indications may be visually distinguishable based on appearance. For instance, indicators 110 may display a countdown of the remaining duration of viability (e.g., in days, hours, minutes, etc.) of samples 106. It should be understood that other configurations for visually distinguishing the various indications are contemplated by this disclosure.

In some instances, samples 106 may need to be transported (or, in other words, delivered) from one geographic location (e.g., a clinic) to one or more other geographic locations (e.g., laboratories, hospitals, other clinics, etc.) for samples 106 to be used for their intended use. For example, samples 106 may be delivered in a vehicle from a clinic to a laboratory by a driver. The driver delivering samples 106 may deliver the samples 106 according to a (pre-determined) delivery schedule. However, due to human errors, logistical oversight, and/or other reasons, the scheduled delivery for samples 106 may result in one or more samples 106 no longer being viable for their intended use. For example, the duration of viability of one or more samples 106 may elapse before the scheduled delivery for samples 106 may be completed.

To address this potential issue in the delivery stage of using samples 106, computing system 104 may determine a delivery recommendation and perform the action of outputting a delivery recommendation to expedite a delivery of the applicable samples. The delivery recommendation may be such that if the applicable samples are delivered in accordance with the delivery recommendation, the applicable samples will more likely be viable for their intended use.

For example, computing system 104 may determine, based on the sample information received from sample containers 102, that the duration of viability of one or more applicable samples (e.g., samples 106A-106C) will elapse prior to the delivery of samples 106. Responsive to this determination, computing system 104 may output a delivery recommendation to the driver delivering samples 106 recommending that the driver modify the scheduled delivery of samples 106 or otherwise use a specific delivery schedule for samples 106.

For example, the delivery recommendation may include a recommended time and/or date for delivering samples 106 that will ensure that the applicable samples are viable for their intended use. In another example, the delivery recommendation may include a recommended destination for the applicable samples. In yet another example, the delivery recommendation may include a recommended route for delivery of the applicable samples. For instance, the delivery recommendation may indicate a route to a first location (e.g., a first laboratory) and then a second location (e.g., a second laboratory), and so on. In some examples, the indicated route may include turn-by-turn directions.

In some examples, the action performed by computing system 104 may be generating notifications (e.g., to a driver, to a technician, to a clinician, etc.) regarding the viability of the applicable samples. For example, the notifications may be generated when the viability of the applicable samples reaches specific thresholds (e.g., remaining duration of viability is 75% of maximum duration of viability, remaining duration of viability is 10% of maximum duration of viability, etc.). The notifications may be issued to anyone involved in the supply chain (e.g., a driver, a lab technician, a clinician, etc.). For example, computing system 104 may generate a notification that the duration of viability will elapse prior to the delivery of samples 106 and issue that notification to a driver delivering samples 106. Additionally or alternatively, the notifications may notify a clinician that a replacement sample is required (e.g., due to the viability of one or more of applicable samples expiring). In yet another example, the notifications may notify a technician that the usage (e.g., testing) of the applicable samples should be prioritized over usage of the other samples due to the remaining viability of the applicable samples. For example, the notifications may notify a technician that unless one or more of the applicable samples are used within 24 hours, the applicable samples will expire and no longer be viable for their intended uses.

It should be understood that computing system 104 may determine one or more actions for increasing the likelihood that the applicable samples will be viable for their intended use. For example, computing system 104 may determine that a first action of causing indicators 110 to indicate a duration of viability for samples 106 in a second action of generating a notification to a driver delivering samples 106 will increase the likelihood that the applicable samples will be viable for their intended use. Computing system 104 may then perform the one or more actions determined to increase the likelihood that the applicable samples will be viable for their intended use.

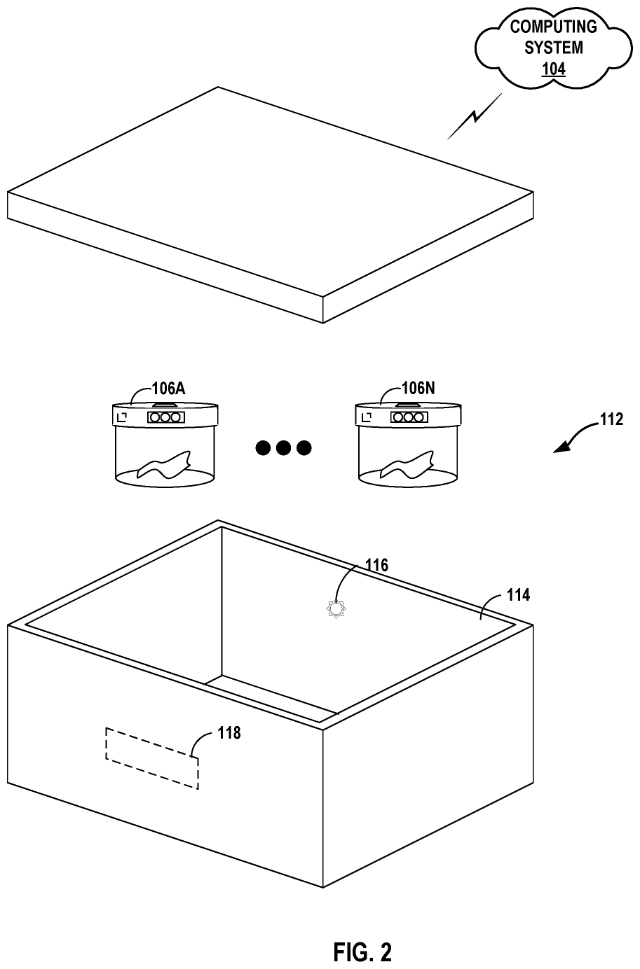

FIG. 2 is a conceptual diagram illustrating an example storage container 112 for storing sample containers 102 in accordance with techniques of this disclosure. As shown in the example of FIG. 2, storage container 112 may define a cavity 114 for storing sample containers 102. In some examples, storage container 112 may include a receptacle, such as a tray, into which sample containers 102 may be inserted. The receptacle may be configured to secure (e.g., via mechanical communication) sample containers 102 to resist excessive movement of sample containers 102 relative to storage container 112.

Because samples 106 may need to be transported from one geographic location (e.g., a clinic) to one or more other geographic locations (e.g., laboratories) for samples 106 to be used for their intended use, samples 106 may be stored in a storage container during delivery, such as storage container 112. Some storage containers may not provide a physical environment conducive to the viability of one or more of samples 106. For example, a cavity of a storage container in which samples 106 are stored may be too cold for the applicable samples of samples 106, potentially reducing the duration of viability of the applicable samples. However, the storage container may not be configured to control the temperature (or any other physical condition) of the cavity of the storage container. As such, some storage containers may be ineffective at ensuring the viability of samples 106 during delivery of samples 106.

To help ensure the viability of samples 106 during delivery of samples 106, storage container 112 may be configured to adjust one or more physical conditions of cavity 114. For example, storage container 112 may include processing circuitry 118 configured to implement functionality and/or execute instructions associated with storage container 112. Processing circuitry 118 may be similar, if not substantially similar, to processing circuitry 109, except for any differences described herein. In some examples, storage container 112 may control, via processing circuitry 118, a heating device or cooling device in thermal communication with storage container 112 to adjust the temperature of cavity 114. Similarly, storage container 112 may control, via processing circuitry 118, a light source 116 to adjust the light level within cavity 114. In some examples, computing system 104 may include processing circuitry 118.

Storage container 112 may further include communication components (not shown for ease of illustration) configured to receive and transmit various types of information. The communication components of storage container 112 may be similar, if not substantially similar, to communication components 120 of sample container 102. For example, storage container 112 may receive, via communication components, sample information from sample containers 102 and transmit, via communication components, the sample information to computing system 104. In this way, storage container 112 may assist in monitoring the viability of samples 106. In another example, storage container 112 may receive, via communication components, information from computing system 104, such as physical conditions (e.g., temperature, light level, etc.) conducive to one or more of samples 106.

In some examples, computing system 104 may, as part of performing the action for increasing the likelihood that one or more applicable samples will be viable for the intended uses of the applicable samples, cause storage container 112 to adjust one or more physical conditions of cavity 114. For example, responsive to receiving and based on information from computing system 104 about the physical conditions conducive to the applicable samples, source container 112 may adjust, via processing circuitry 118, the temperature, light level, and/or the like within cavity 114 to achieve the physical conditions determined by computing system 104 to be conducive to the applicable samples.

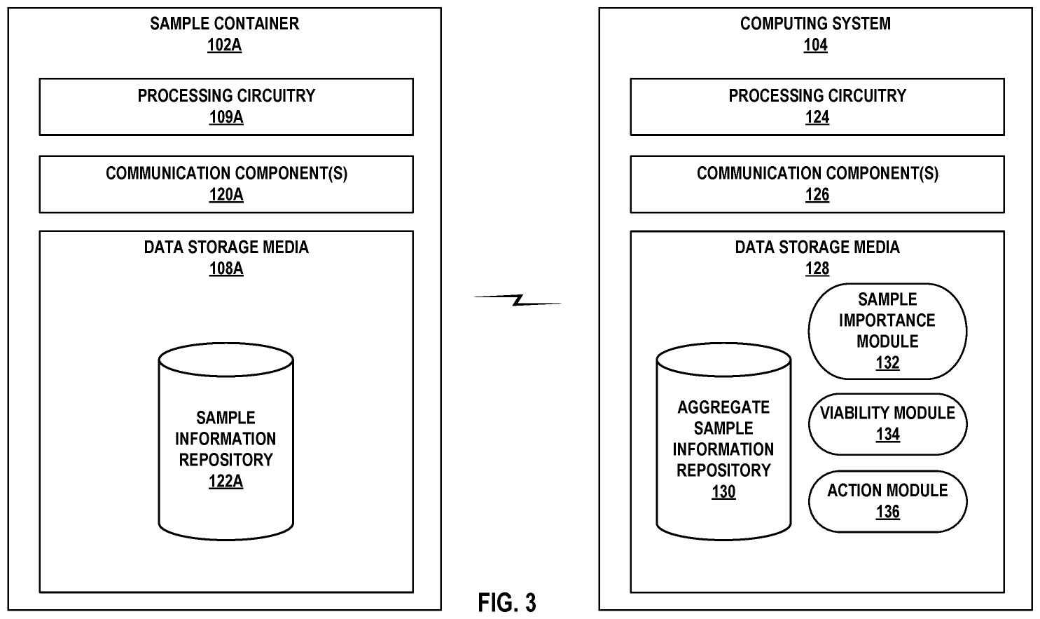

FIG. 3 is a block diagram illustrating example system 100 for monitoring and increasing viability of applicable samples in accordance with techniques of this disclosure. As shown in FIG. 3, system 100 may include sample container 102A and computing system 104. Although only sample container 102A of sample containers 102 is illustrated in FIG. 3, it should be understood that system 100 may include other sample containers, such as sample container 102B, sample container 102C, and/or the like. Further, it should be understood that any description of sample container 102A may apply equally to the others (e.g., sample container 102B, sample container 102C, etc.).

As shown in FIG. 3, sample container 102A may include processing circuitry 109A (described in greater detail with respect to FIG. 1), communication components 120A (described in greater detail with respect to FIG. 1), and data storage media 108A (described in greater detail with respect to FIG. 1). Similarly, computing system 104 may include processing circuitry 124, communication components 126, data storage media 128. Processing circuitry 124 may be similar, if not substantially similar, to processing circuitry 109, except for any differences described herein. Communication components 126 may be similar, if not substantially similar, to communication components 120, except for any differences described herein. Data storage media 128 may be similar, not substantially similar, to data storage media 108, except for any differences described herein. Data storage media 128 may include aggregate sample information repository 130, sample importance module 132, viability module 134, and action module 136.

As further shown in FIG. 3, data storage media 108A may include a sample information repository 122A. Sample information repository 122A may store sample information about sample 106A stored inside sample container 102A. For example, sample information repository 122A may store information such as the type of sample 106A (e.g., the name of sample 106A, the properties of sample 106A, etc.), the intended use of sample 106A (e.g., the test to be performed on sample 106A), temperature conditions (e.g., data about the temperature at which sample 106A is to be stored, data about the temperature at which sample 106A has been stored, etc.), centrifuge detection (e.g., data about whether a centrifuge has been applied to sample 106A), a duration of viability (of sample 106A), light sensitivity (of sample 106A), properties of sample container 102A (which is storing sample 106A), multi-variant date and time (e.g., origination of sample 106A, the expiration date of sample container 102A, entries documenting when sample container 102A has been moved from one geographic location (e.g., a first laboratory) to another (e.g., a second laboratory), etc.), a sample importance of sample 106A, electronic health record (EHR) data (e.g., name of the patient from whom sample 106A was collected, the date of birth of that patient, the name of the clinic that patient visits, the name of that patient's primary doctor, etc.), expected end-to-end process time (e.g., beginning with collection of sample 106A and ending with utilization of sample 106A), and/or the like. Sample container 102A may transmit, via communication components 120A, the sample information stored in sample information repository 122A to computing system 104 (over a network).

Each of sample containers 102 may include respective data storage media 109 that store corresponding sample information repositories. For example, as shown in FIG. 3, data storage media 108A of sample container 102A may store sample information repository 122A. Each of sample containers 102 may transmit, via corresponding communication components 120, sample information about each of samples 106 stored in sample containers 102 to computing system 104. For example, sample container 102A may transmit, via communication components 120A, sample information about sample 106A stored in sample information repository 122A to computing system 104. Computing system 104 may store the sample information from each of sample containers 102 in an aggregate sample information repository 130.

In some examples, the sample information stored in aggregate sample information repository 130 may include the sample importance score for each of samples 106. In examples where the sample information stored in aggregate sample information repository 130 does not include the sample importance scores, computing system 104 may determine the sample importance scores of each of samples 106 in order to classify samples with relatively high sample importance scores 106 as applicable samples. Sample importance module 132 may determine the sample importance scores based on various factors, each of which may be stored in aggregate sample information repository 130. For example, the factors may include the medical importance of the results from using the sample, the urgency of the results from using the sample, the collection importance of the sample (e.g., based on the difficulty of obtaining the sample, the availability of the sample, etc.), the handling importance (e.g., based on the sensitivity and/or fragility of the sample), and/or the like. Sample importance module 132 may calculate the sample importance score using an algorithm, a mathematical model, and/or other techniques.

Responsive to determining the sample importance scores for each of samples 106, sample importance module 132 may classify the set of samples 106 with relatively high sample importance scores as applicable samples. For example, if the sample importance scores range from 0 to 10, where zero is indicative of a low importance, and 10 is indicative of a high importance, sample importance module 132 may classify the set of samples 106 with sample importance scores above a threshold value (e.g., 5, 7, 9, an average sample importance score of samples 106, etc.) to be applicable samples and the rest as not being applicable samples. In some examples, sample importance module 132 may rank samples 106 by the respective sample importance scores and classify a pre-determined number and/or proportion of samples of samples 106 as applicable samples based on the ranking. For example, sample importance module 132 may classify the 25% of samples 106 that have the highest sample importance scores according to the ranking as applicable samples, and classify the remaining 75% as not applicable samples.

Responsive to sample importance module 132 classifying the applicable samples, computing system 104 may use viability module 134 to determine an action for increasing the likelihood the one or more applicable samples will be viable for the intended uses of the applicable samples. Viability module 134 may determine the action based on the sample information and aggregate sample repository 130. For example, if sample importance module 132 classifies samples 106A-106C as the applicable samples, viability module 134 may obtain sample information from aggregate sample repository 130 indicating that the duration of viability of samples 106A-106C is greatest when samples 106A-106C are stored in a frozen temperature range. Accordingly, viability module 134 may determine that setting (e.g., adjusting or maintaining) the temperature of a cavity (e.g., cavity 114) of a storage container (e.g., storage container 112) in which samples 106A-106C are stored will increase the likelihood of the applicable samples being viable for their intended uses.

Action module 136 of computing system 104 may perform the action determined by viability module 134. For example, if viability module 134 determines that the temperature of cavity 114 in which applicable samples 106A-106C are stored needs to be adjusted from an ambient temperature range to a frozen temperature range, action module 136 may send, via communication components 126, a request/command to storage container 112 to decrease the temperature inside cavity 114 until it is within the frozen temperature range. Responsive to receiving the request/command from computing system 104, storage container 112, via processing circuitry 118, may control the cooling device in thermal communication with cavity 114 to cool cavity 114 accordingly.

As described above, computing system 104 may determine and perform actions in addition or alternative to adjusting one or more physical conditions of an environment in which sample containers 102, and thus samples 106, are stored. For example, viability module 134 may determine that controlling indicators 110 to output an indication regarding viability of samples 106, generating driving recommendations, modifying driving routes and/or schedules, generating notifications (e.g., regarding remaining duration of viability of sample 106), and/or the like will increase the likelihood of the applicable samples being viable for their intended uses. Action module 136 may then perform one or more of these actions.

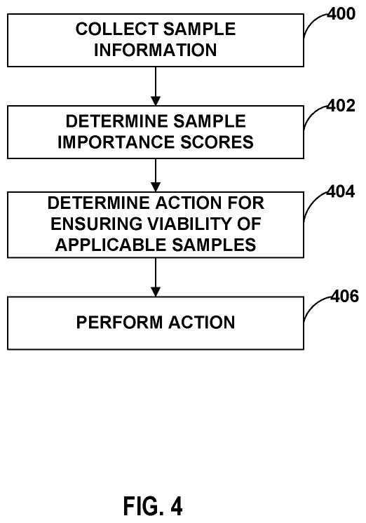

FIG. 4 is a flowchart illustrating an exemplary operation of an example system in accordance with techniques of this disclosure. As shown in FIG. 4, computing system 104 may collect sample information about samples 106 stored in sample containers 102 (400). In some examples, computing system 104 may receive the sample information from sample containers 102. For example, sample containers 102 may include data storage media 109 that store the sample information about samples 106 in sample information repositories 122. Sample containers 102 may transmit the sample information to computing system 104 via communication components 120 (e.g., over a network). Computing system 104 may then store the sample information from one or more of sample containers 102 in aggregate sample information repository 132.

Based on the sample information in aggregate sample information repository 132, sample importance module 132 may determine the respective sample importance scores for samples 106 in order to classify one or more of samples 106 as applicable samples (402). For example, sample importance module 132 may determine (via processing circuitry 124) that sample 106A has a relatively high sample importance score because the medical importance of the results from using sample 106A is high, the urgency of the results from using sample 106A is high, the collection importance of sample 106A is high because obtaining sample 106A is difficult, and the handling importance of sample 106A is high because of the sensitivity of (the duration of viability of) sample 106A to the physical conditions of the environment in which sample 106A is stored. Accordingly, sample importance module 132 may classify sample 106A as an applicable sample.

In another example, sample importance module 132 may determine that sample 106B has a relatively low sample importance score because the medical importance of the results from using sample 106B is low, the urgency of the results from using sample 106B is low, the collection importance of sample 106B is low because obtaining sample 106B is easy, the handling importance of sample 106B is low because of the sensitivity of (the duration of viability of) sample 106B to the physical conditions of the environment in which sample 106B is stored. Accordingly, sample importance module 132 may not classify sample 106B as an applicable sample. In this way, sample importance module 132 may classify each of samples 106 as applicable samples or not as applicable samples.

Responsive to sample importance module 132 determining the applicable samples of samples 106, viability module 134 may determine an action that will increase the likelihood of the applicable samples being viable for their intended uses, even if doing so will decrease the likelihood of samples that are not applicable samples being viable for their intended uses (404).

By determining which samples of samples 106 are applicable samples in this way, computing system 104 may determine that the applicable samples have higher importance than other samples of samples 106 and that the viability of the applicable samples should be ensured, even at the expense of the viability of other samples. For example, if sample importance module 132 classifies samples 106A-106C as the applicable samples, viability module 134 may obtain sample information from aggregate sample repository 130 indicating that the duration of viability of samples 106A-106C is greatest when samples 106A-106C are stored in a frozen temperature range. Accordingly, viability module 134 may determine that setting (e.g., adjusting or maintaining) the temperature of a cavity (e.g., cavity 114) of a storage container (e.g., storage container 112) in which samples 106A-106C are stored will increase the likelihood of the applicable samples being viable for their intended uses.

In some examples, viability module 134 may determine a delivery recommendation and perform the action of outputting a delivery recommendation to expedite a delivery of the applicable samples. The delivery recommendation may be such that if the applicable samples are delivered in accordance with the delivery recommendation, the applicable samples will more likely be viable for their intended use.

In some examples, viability module 134 may determine one or more actions for increasing the likelihood that the applicable samples will be viable for their intended use. For example, viability module 124 may determine that a first action of causing indicators 110 to indicate a duration of viability for samples 106 in a second action of generating a notification to a driver delivering samples 106 will increase the likelihood that the applicable samples will be viable for their intended use.

Action module 136 of computing system 104 may perform the one or more actions determined by viability module 134 (406). For example, if viability module 134 determines that the temperature of cavity 114 in which applicable samples 106A-106C are stored needs to be adjusted from an ambient temperature range to a frozen temperature range, action module 136 may send, via communication components 126, a request/command to storage container 112 to decrease the temperature inside cavity 114 until it is within the frozen temperature range. Responsive to receiving the request/command from computing system 104, storage container 112, via processing circuitry 118, may control the cooling device in thermal communication with cavity 114 to cool cavity 114 accordingly to increase the likelihood that the applicable samples will be viable for their intended use.

Action module 136 may perform one or more actions in addition to or alternative to controlling the physical conditions of the environment in which samples 106 are stored. For example, action module 136 may perform the action of communicating information to personnel involved in the supply chain for samples 106, potentially enabling the personnel to respond based upon the communicated information. In this way, the action of communicating information performed by action module 136 may increase the likelihood that the applicable samples will be viable for their intended use.

In some examples, action module 136 communicates information by causing sample containers 102 to output one or more indications. For example, action module 136 may cause indicators 110 to output an indication regarding viability of samples 106 contained in sample containers 102 for the intended use of samples 106. In some examples, indicators 110 may be light sources configured to display a plurality of indications. For example, indicators 110 may display a first indication, a second indication, and a third indication. The first indication may correspond to a (remaining) duration of viability that is 90-100% of the maximum duration (or, in other words, the total expected duration) of viability of samples 106. The second indication may correspond to a duration of viability that is 10-90% of the maximum duration of viability of samples 106. The second indication may correspond to a duration of viability that is 0-10% of the maximum duration of viability of samples 106.

In some examples, responsive to viability module 134 determining that the duration of viability of one or more applicable samples (e.g., samples 106A-106C) will elapse prior to the delivery of samples 106, action module 136 may output a delivery recommendation to relevant personnel in the supply chain (e.g., a driver delivering samples 106) recommending that the scheduled delivery of samples 106 be modified or that a specific (modified) delivery schedule for samples 106 be used.

For example, the delivery recommendation may include a recommended time and/or date for delivering samples 106 that will ensure that the applicable samples are viable for their intended use. In another example, the delivery recommendation may include a recommended destination for the applicable samples. In yet another example, the delivery recommendation may include a recommended route for delivery of the applicable samples. For instance, the delivery recommendation may indicate a route to a first location (e.g., a first laboratory) and then a second location (e.g., a second laboratory), and so on. In some examples, the indicated route may include turn-by-turn directions.

In some examples, the action performed by action module 136 may be generating notifications (e.g., to a driver, to a technician, to a clinician, etc.) regarding the viability of the applicable samples. For example, the notifications may be generated when the viability of the applicable samples reaches specific thresholds (e.g., remaining duration of viability is 75% of maximum duration of viability, remaining duration of viability is 10% of maximum duration of viability, etc.). The notifications may be issued to anyone involved in the supply chain (e.g., a driver, a lab technician, a clinician, etc.). For example, action module 136 may generate a notification that the duration of viability will elapse prior to the delivery of samples 106 and issue that notification to a driver delivering samples 106. Additionally or alternatively, the notifications may notify a clinician that a replacement sample is required (e.g., due to the viability of one or more of applicable samples expiring). In yet another example, the notifications may notify a technician that the usage (e.g., testing) of the applicable samples should be prioritized over usage of the other samples due to the remaining viability of the applicable samples. For example, the notifications may notify technician that unless one or more of the applicable samples are used within 24 hours, the applicable samples will expire and no longer be viable for their intended uses.

By way of example, and not limitation, such computer-readable storage media can comprise RAM, ROM, EEPROM, CD-ROM or other optical disk storage, magnetic disk storage, or other magnetic storage devices, flash memory, or any other storage medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage mediums and media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable medium.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A system comprising:
a plurality of sample containers, wherein:
each respective sample container of the plurality of sample containers defines a cavity to contain a respective sample of a plurality of samples, and
each respective sample container of the plurality of sample containers comprises one or more data storage media configured to store sample information relating to the respective sample contained in each sample container, the sample information comprising data indicating:
a sample type;
an intended use of the sample contained within the respective sample container; and
a sample collection time; and
a computing system configured to:
receive the sample information from the sample containers; and
determine, based on the sample information, an action for increasing a likelihood that one or more applicable samples of the plurality of samples will be viable for the intended uses of the applicable samples; and
perform the action.

2. The system of claim 1, wherein:
for each respective sample container of the plurality of sample containers, the respective sample container comprises an indicator configured to output an indication regarding viability of the sample contained in the respective sample container for the intended use of the sample contained in the sample container, and
the computing system is configured such that, as part of performing the action, the computing system causes the indicators to output the indications.

3. The system of claim 2, wherein the indicator is a light source configured to display a plurality of indications, wherein each of the indications corresponding to a different duration of viability of the sample contained in the respective sample container.

4. The system of claim 1, wherein for each at least one sample container of the plurality of sample containers, the computing system comprises processing circuitry included in a component of the sample container.

5. The system of claim 1, further comprising a storage container defining a cavity for storing the plurality of sample containers, wherein the storage container is configured to adjust one or more physical conditions of a plurality of physical conditions of the cavity of the storage container.

6. The system of claim 5, wherein the computing system comprises processing circuitry included in the storage container.

7. The system of claim 5, wherein the computing system is configured such that, as part of performing the action, the computing system causes the storage container to adjust one or more physical conditions of a plurality of physical conditions of the cavity of the storage container.

8. The system of claim 5, wherein the plurality of physical conditions comprises a temperature of the cavity and a light level within the cavity.

9. The system of claim 1, wherein the computing system is configured such that, as part of performing the action, the computing system outputs a delivery recommendation to expedite a delivery of the applicable samples, wherein the delivery recommendation comprises one or more of a recommended destination for the applicable samples, a recommended time of delivering the applicable samples, or a route for delivery of the applicable samples.

10. The system of claim 1, wherein the computing system is configured such that, as part of performing the action, the computing system generates a notification regarding viability of the applicable samples.

11. The system of claim 1,
wherein the computing system determines a sample importance for each sample of the plurality of samples based on the sample information of the sample containers, and
wherein the computing system determines the applicable samples based on the sample importance for each sample.

12. The system of claim 11, wherein the computing system is configured such that, as part of determining the applicable samples, the computing system determines that the applicable samples have higher importance than other samples in the plurality of samples.

13. A method comprising:
receiving, by a computing system, sample information from each respective sample container of a plurality of sample containers, wherein:
each respective sample container defines a cavity to contain a respective sample of a plurality of samples, and
each respective sample container of the plurality of sample containers comprises one or more data storage media configured to store the sample information relating to the respective sample contained in each sample container, the sample information comprising data indicating:
a sample type;
an intended use of the sample contained within the respective sample container; and
a sample collection time;
determining, by the computing system and based on the sample information, an action for increasing a likelihood that one or more applicable samples of the plurality of samples will be viable for the intended uses of the applicable samples; and
performing, by the computing system, the action.

14. The method of claim 13, wherein:
for each respective sample container of the plurality of sample containers, the respective sample container comprises an indicator configured to output an indication regarding viability of the sample contained in the respective sample container for the intended use of the sample contained in the sample container, and
the computing system is configured such that, as part of performing the action, the computing system causes the indicators to output the indications.

15. The method of claim 13, wherein for each at least one sample container of the plurality of sample containers, the computing system comprises processing circuitry included in a component of the sample container.

16. The method of claim 13, further comprising storing the plurality of sample containers in a storage container defining a cavity for storing the plurality of sample containers, wherein the storage container is configured to adjust one or more physical conditions of a plurality of physical conditions of the cavity of the storage container.

17. The method of claim 13, wherein the computing system is configured such that, as part of performing the action, the computing system causes the storage container to adjust one or more physical conditions of a plurality of physical conditions of the cavity of the storage container.

18. The method of claim 13, wherein the plurality of physical conditions comprises a temperature of the cavity, a humidity of the cavity, and a light level within the cavity.

19. The method of claim 13, wherein the computing system is configured such that, as part of performing the action, the computing system outputs a delivery recommendation to expedite a delivery of the applicable samples, wherein the delivery recommendation comprises one or more of a recommended destination for the applicable samples, a recommended time of delivering the applicable samples, or a route for delivery of the applicable samples.

20. The method of claim 13, wherein the computing system determines a sample importance for each sample of the plurality of samples based on the sample information of the sample containers, and
wherein the computing system determines the applicable samples based on the sample importance for each sample.
